(19) United States
(12) Patent Application Publication
(10) Pub. No.: US 2023/0191068 A1
(43) Pub. Date: Jun. 22, 2023

(54) VAPORIZATION CONFIGURATIONS FOR BREATHING GASES HUMIDIFIER

(71) Applicant: Covidien LP, Mansfield, MA (US)

(72) Inventors: Donald J. NOVKOV, Encinitas, CA (US); Matthew J. PHILLIPS, Carlsbad, CA (US)

(73) Assignee: Covidien LP, Mansfield, MA (US)

(21) Appl. No.: 18/058,489

(22) Filed: Nov. 23, 2022

Related U.S. Application Data
(60) Provisional application No. 63/292,562, filed on Dec. 22, 2021.

(51) Int. Cl. A61M 16/10; A61M 16/16

(52) U.S. Cl. CPC A61M 16/1095; A61M 16/16

(57) ABSTRACT

A humidification system including a conduit for carrying breathing gases, a pump to pressurize a liquid for injection into breathing gases, a liquid-injection nozzle protruding at least partially into the conduit and configured to inject liquid, pressurized by the pump, into the conduit; and a heated surface protruding into the conduit and positioned to vaporize the liquid injected by the nozzle, wherein the heated surface crosses a flow path of the breathing gases flowing through the conduit.

### CROSS-REFERENCE TO RELATED APPLICATIONS

This application claims the benefit of U.S. Provisional Application No. 63/292,562 filed on Dec. 22, 2021, entitled “Vaporization Configurations for Breathing Gases Humidifier,” which is incorporated herein by reference in its entirety.

### INTRODUCTION

Medical ventilator systems have long been used to provide ventilatory and supplemental oxygen support to patients. These ventilators typically comprise a source of pressurized oxygen which is fluidly connected to the patient through a conduit or tubing. Some ventilators are used with humidifiers to humidify the gas delivered to the patient to improve patient adherence and comfort.

### SUMMARY

In an aspect, the technology relates to a humidification system including a conduit for carrying breathing gases; a pump to pressurize a liquid for injection into breathing gases; a liquid-injection nozzle protruding at least partially into the conduit and configured to inject liquid, pressurized by the pump, into the conduit; and a heated surface protruding into the conduit and positioned to vaporize the liquid injected by the nozzle, wherein the heated surface crosses a flow path of the breathing gases flowing through the conduit.

In another example, the technology relates to a heated surface is a ramped surface protruding from an inner wall of the conduit. In a further example, the ramped surface has a ramp angle from the conduit wall between 10 degrees and 50 degrees. In a still further example, the ramped surface has a width of at least 30% of a diameter of the conduit. In yet another example, the nozzle is configured to inject the liquid in one of a jet spray pattern or fan beam spray pattern. In still another example, the heated surface is substantially orthogonal to the flow of breathing gases. In still yet another example, the heated surface is perforated to allow the breathing gases to flow through the perforations.

In another example, the heated surface has a concave portion facing the flow of breathing gases and the nozzle, wherein the concave portion is positioned to receive and vaporize the injected liquid. In yet another example, the heated surface has a convex portion facing the flow of gases and the nozzle, wherein the convex portion is positioned relative to the nozzle to receive and vaporize the injected liquid. In still another example, the heated surface is at least one of: parabola-shaped, bowl-shaped, conic, frustroconic, substantially cylindrical, partially cylindrical. In still yet another example, a portion of the heated surface is positioned downstream of the nozzle and another portion of the heated surface is positioned upstream of the nozzle. In another example, the heated surface substantially encapsulates the nozzle.

In another aspect, the technology relates to a humidification system that includes a conduit for carrying breathing gases; a pump to pressurize a liquid for injection into breathing gases; a liquid-injection nozzle protruding at least partially into the conduit and configured to inject liquid, pressurized by the pump, into the conduit; and a ramped heated surface protruding into the conduit from an interior surface of the conduit and positioned to vaporize at least a portion of the liquid injected by the nozzle, wherein the ramped heated surface protrudes into the conduit at a ramp angle between 20-90 degrees.

In an example, the ramped heated surface has a height between 10%-70% of an inner diameter of the conduit, wherein the height of the ramped heated surface is the maximum height of the heated surface from the interior surface of the conduit. In a further example, the nozzle has a nozzle height between 20%-80% of the height of the ramped heated surface, wherein the nozzle height is a height above the interior surface of the conduit to a center point of the nozzle from where the liquid is injected by the nozzle. In still another example, the ramped surface has a width of at least 30% of a diameter of the conduit. In yet another example, the nozzle is configured to inject the liquid in a direction opposite a direction of a flow path of the breathing gases. In still yet another example, the ramped heated surface is positioned upstream, with respect to the flow path of breathing gases, from the nozzle. In a further example, the nozzle has an upward nozzle angle between 10-30 degrees.

In another aspect, the technology relates to a method for humidifying ventilator-delivered breathing gases. The method includes heating a surface protruding into a conduit, wherein the heated surface is non-parallel with the flow of breathing gases through the conduit; pressurizing, by a pump, a liquid; injecting the pressurized liquid through a nozzle protruding into the conduit, wherein the injected liquid impinges the heated surface; and vaporizing, by the heated surface, the injected liquid. These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the present disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims appended hereto.

FIG. 1 is schematic diagram illustrating an example medical ventilation system.

FIG. 2 is schematic diagram illustrating another example medical ventilation system.

FIG. 3 is a partial, cross-sectional schematic diagram illustrating an example humidifier.

FIG. 4A depicts a perspective view of a partial, cross-sectional diagram illustrating a configuration for a nozzle and a heated surface of a humidifier.

FIG. 4B depicts a side view of the configuration depicted in FIG. 4A.

FIG. 4C depicts another example configuration with a different nozzle angle.

FIG. 4D depicts another example configuration with a curved heated surface.

FIG. 5A depicts an example configuration for a nozzle and a heated surface of a humidifier.

FIG. 5B depicts another example configuration for a nozzle and a heated surface of a humidifier.

FIG. 5C depicts another example configuration for a nozzle and a heated surface of a humidifier.

FIG. 5D depicts another example configuration for a nozzle and a heated surface of a humidifier.

FIG. 6A depicts another example configuration for a nozzle and a heated surface of a humidifier.

FIG. 6B depicts another example configuration for a nozzle and a heated surface of a humidifier.

FIG. 7 depicts another example configuration for a nozzle and a heated surface of a humidifier.

FIG. 8 depicts an example perforation pattern for a heated surface.

FIG. 9 depicts an example method for humidifying breathing gases from a ventilator.

### DETAILED DESCRIPTION

Although the techniques introduced above and discussed in detail below may be implemented for a variety of medical devices, the present disclosure will discuss the implementation of these techniques in the context of a medical ventilator for use in providing ventilation support to a human patient. A person of skill in the art will understand that the technology described in the context of a medical ventilator for human patients could be adapted for use with other systems such as ventilators for non-human patients and general gas transport systems.

Medical ventilators are used to provide breathing gases to a patient who may otherwise be unable to breathe sufficiently. In modern medical facilities, pressurized air and oxygen sources are often available from wall outlets. Accordingly, ventilators may provide pressure regulating valves (or regulators) connected to centralized sources of pressurized air and pressurized oxygen. The regulating valves function to regulate flow so that respiratory gases having a desired concentration of oxygen are supplied to the patient at desired pressures and rates. Ventilators capable of operating independently of external sources of pressurized air are also available.

While operating a ventilator, it is desirable to control the percentage of oxygen in the gases supplied by the ventilator to the patient. Further, some ventilators are used with humidifiers to humidify the breathing gases delivered to the patient to improve patient adherence and comfort. However, some humidifiers often over humidify the delivered breathing gases leading to an accumulation of water in the patient circuit or within the lungs of patient, referred to herein as “rainout.” The accumulated water in the patient circuit can interfere with circuit sensors and/or filters and can increase the chances of patient infection, such as pneumonia. Accordingly, the accumulated water must be removed or cleared from the patient circuit, and over-humidification leading to rainout is problematic with current ventilator humidifiers. Under humidification is also problematic, particularly in low-gas flow ventilator operating conditions, because under humidification for prolonged periods can result in airway damage due to dryness and other patient harm.

Humidifiers that may be more prone to rainout generally include a reservoir of water and a large heating plate that heats the reservoir of water. As the reservoir of water is heated, the evaporated water flows into the patient circuit to humidify the gas that is being delivered to the patient. In such systems, the amount of humidification introduced into the circuit is directly tied with amount of heat introduced by heating plate. That is, the amount of water introduced into the patient circuit cannot be separately controlled from the amount of heat introduced into the patient circuit.

Accordingly, the current disclosure describes systems and methods for humidifying ventilator delivered breathing gases that reduces and/or prevents rainout. The present technology directly controls the amount of water or liquid that is injected into the breathing circuit, which helps prevent rainout from over-humidification. More specifically, the present technology injects a pressurized liquid through a nozzle. The pressurized liquid may be injected in a variety of patterns, including a jet, a full cone, a hollow cone, a fan shape, etc. The liquid may be injected as a stream or as atomized droplets. The pressurized liquid is injected such that it impinges a heated surface, protruding into a conduit carrying breathing gases, that evaporates the injected liquid. The water vapor then mixes with breathing gases to form humidified breathing gases that are carried to the patient through the remainder of the breathing circuit.

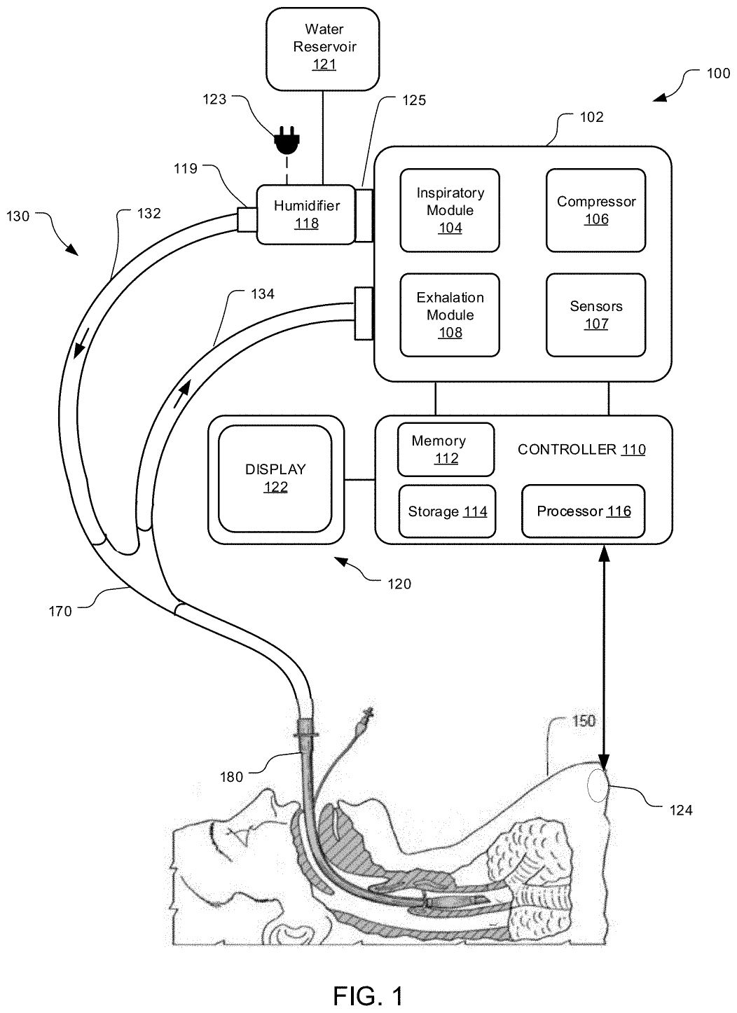

FIG. 1 is a diagram illustrating a first aspect of an example ventilation system or ventilator 100 connected to a human patient 150. Ventilator 100 includes a pneumatic system 102 (also referred to as a pressure generating system 102) for circulating breathing gases to and from patient 150 via the ventilation tubing system 130, which couples the patient 150 to the pneumatic system 102 via a patient interface 180, which may be an invasive patient interface (e.g., endotracheal tube, as shown) or a non-invasive patient interface (e.g., nasal mask or nasal prongs, not shown).

Ventilation tubing system 130 (or patient circuit 130) may be a two-limb (shown) or a one-limb circuit for carrying gases to and from the patient 150. In a two-limb aspect, a fitting, typically referred to as a “wye-fitting” 170, may be provided to couple the patient interface 180 to an inspiratory limb 132 and an expiratory limb 134 of the ventilation tubing system 130.

Pneumatic system 102 may be configured in a variety of ways. In the present example, pneumatic system 102 includes an exhalation module 108 coupled with the exhalation limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory port 125 to inspiratory limb 132. The inspiratory module 104 is configured to deliver breathing gases to the patient 150 according to prescribed ventilatory settings. In some aspects, inspiratory module 104 is configured to provide ventilation according to various breath types, e.g., via volume-control, pressure-control, proportional assist control, or via any other suitable breath types. The exhalation module 108 is configured to release gases from the patient's lungs according to prescribed ventilatory settings. Specifically, exhalation module 108 is associated with and/or controls an exhalation valve for releasing gases from the patient 150.

The ventilator 100 may also include one or more sensors 107 communicatively coupled to ventilator 100. The sensors 107 may be located in the pneumatic system 102, ventilation tubing system 130, and/or on the patient 150. FIG. 1 illustrates an example of a sensor 107 in pneumatic system 102. Sensors 107 may communicate with various components of ventilator 100, e.g., pneumatic system 102, other sensors 107, processor 116, humidifier 118, heating tube 119, and/or any other suitable components and/or modules. A module as used herein refers to memory, one or more processors, storage, and/or other components of the type found in command and control computing devices.

In one aspect, sensors 107 generate output and send this output to pneumatic system 102, other sensors 107, processor 116, controller 110, humidifier 118, heating element of heating tube 119, and/or any other suitable components and/or modules. Sensors 107 may employ any suitable sensory or derivative technique for monitoring one or more patient parameters or ventilator parameters associated with the ventilation of a patient 150. Sensors 107 may detect changes in patient parameters indicative of patient triggering, for example. In other examples, the sensor 107 may include a humidity sensor, a temperature sensor, a combined temperature/humidity sensor, and/or inspiratory flow sensor. In some aspects, the humidity sensor determines the humidity and temperature of the breathing gas. In other aspects, the inspiratory flow sensor determines the inspiratory flow rate of the breathing gas.

The sensors 107 may include a thermometer 124. The thermometer 124 may be placed on or in the patient 150. The thermometer 124 may be an internal thermometer, such as a rectal thermometer, or an external thermometer. In some examples, the thermometer 124 may be placed near the lungs or the airways of the patient to more accurately identify the temperature of the lungs and airways of the patient, which may differ from the temperature of other portions of the patient due to localized temperature changes. Thus, the measured temperature may be more accurate for use in setting breathing gas temperature and/or humidity to prevent rainout. As an example, the thermometer 124 may be placed on or in a portion of patient interface that is intended be inside the patient in use, such as a tracheal tube or endotracheal tube 180. The thermometer 124 may be integrated into the endotracheal tube 180 to allow for communication of the temperature measurements back to the ventilator 100 and/or humidifier 118. For instance, wired or wireless components may be integrated into the endotracheal tube 180 to allow for communication of data. To more accurately measure the temperature of the lungs, the thermometer 124 may also be placed towards the distal end (e.g., furthest point away from the ventilator) of the endotracheal tube 180. In some examples the thermometer 124 (or the temperature sensing element of the thermometer 124) may be placed within 8 cm of the distal end of the endotracheal tube 180.

Both external and internal thermometers are capable of measuring an internal temperature of the patient. The thermometer 124 may also include an infra-red thermometer to measure the temperature of the patient 150 without contacting the patient. The thermometer 124 may be in communication with the humidifier 118 or other components of the ventilator 100 via a wired or wireless connection. The thermometer 124 may then communicate the temperature measurements of the patient to the humidifier 118 or other components of the ventilator for use in determining humidification settings as discussed further herein.

Sensors 107 may be placed in any suitable location, e.g., within the ventilatory circuitry or other devices communicatively coupled to the ventilator 100. Further, sensors 107 may be placed in any suitable internal location, such as, within the ventilatory circuitry or within components or modules of ventilator 100. For example, sensors 107 may be coupled to the inspiratory and/or exhalation modules for detecting changes in, for example, circuit pressure and/or flow. In other examples, sensors 107 may be affixed to the ventilatory tubing or may be embedded in the tubing itself. According to some aspects, sensors 107 may be provided at or near the lungs (or diaphragm) for detecting a pressure in the lungs. Additionally or alternatively, sensors 107 may be affixed or embedded in or near wye-fitting 170 and/or patient interface 180. Indeed, any sensory device useful for monitoring changes in measurable parameters during ventilatory treatment may be employed in accordance with aspects described herein.

As should be appreciated, with reference to the Equation of Motion, ventilatory parameters are highly interrelated and, according to aspects, may be either directly or indirectly monitored. That is, parameters may be directly monitored by one or more sensors 107, as described above, or may be indirectly monitored or estimated/calculated using a model, such as a model derived from the Equation of Motion:

Target Airway Pressure(t)=Eₚ∫Qₚdt+QₚRₚ−Patient Effort(t)

The pneumatic system 102 may include a variety of other components, including mixing modules, valves, tubing, accumulators, filters, humidifier 118, heating tube 119, water reservoir 121, etc. In other aspects, these other components are located outside of the pneumatic system 102, such as the mixing modules, valves, tubing, accumulators, filters, humidifier 118, heating tube 119, water reservoir 121, etc.

Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.). In some aspects, the controller in electronic communication with and/or operatively coupled to a humidifier 118 and/or a heating tube 119. For example, the controller 110 of the ventilator 100 may send an inspiratory flow command, inspiratory flow measurements, and/or temperature or humidity measurements of the breathing gases to the humidifier 118 and/or a heating tube 119.

In one aspect, the operator interface 120 of the ventilator 100 includes a display 122 communicatively coupled to ventilator 100. Display 122 provides various input screens, for receiving clinician input, and various display screens, for presenting useful information to the clinician. In one aspect, the display 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 100 may be provided, for instance by a wheel, keyboard, mouse, or other suitable interactive device. Thus, operator interface 120 may accept commands and input through display 122. Display 122 may also provide useful information in the form of various ventilatory data regarding the physical condition of a patient 150. The useful information may be derived by the ventilator 100, based on data collected by a processor 116, and the useful information may be displayed to the clinician in the form of graphs, wave representations, pie graphs, text, or other suitable forms of graphic display. For example, patient data may be displayed on the GUI and/or display 122. Additionally or alternatively, patient data may be communicated to a remote monitoring system coupled via any suitable means to the ventilator 100. In one aspect, the display 122 may display one or more of a flow rate, a relative humidity of the breathing gases, a temperature of the breathing gases, a selected breath type, a humidifier on or a humidifier off status, etc.

Controller 110 may include memory 112, one or more processors 116, storage 114, and/or other components of the type commonly found in command and control computing devices. The memory 112 includes non-transitory, computer-readable storage media that stores and/or encodes software (such as computer executable instruction) that is executed by the processor 116 and which controls the operation of the ventilator 100. In an aspect, the memory 112 includes one or more solid-state storage devices such as flash memory chips. In an alternative aspect, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

As illustrated by FIG. 1, the ventilator 100 also includes a humidifier 118 located upstream of the patient interface 180 to humidify the breathing gases delivered to the patient 150. The humidifier 118 my included a heated surface configured to evaporate liquids that are injected by a nozzle of the humidifier 118, as discussed further below. In some examples, the humidifier 118 may also include the heating tube 119, while in other aspects, the heating tube 119 is separate from and independent of the humidifier 118. In other examples, however, the heated tube 119 may be omitted where a heated surface for evaporating the injected liquid is included in the humidifier 118 and or another portion of the breathing circuit 130.

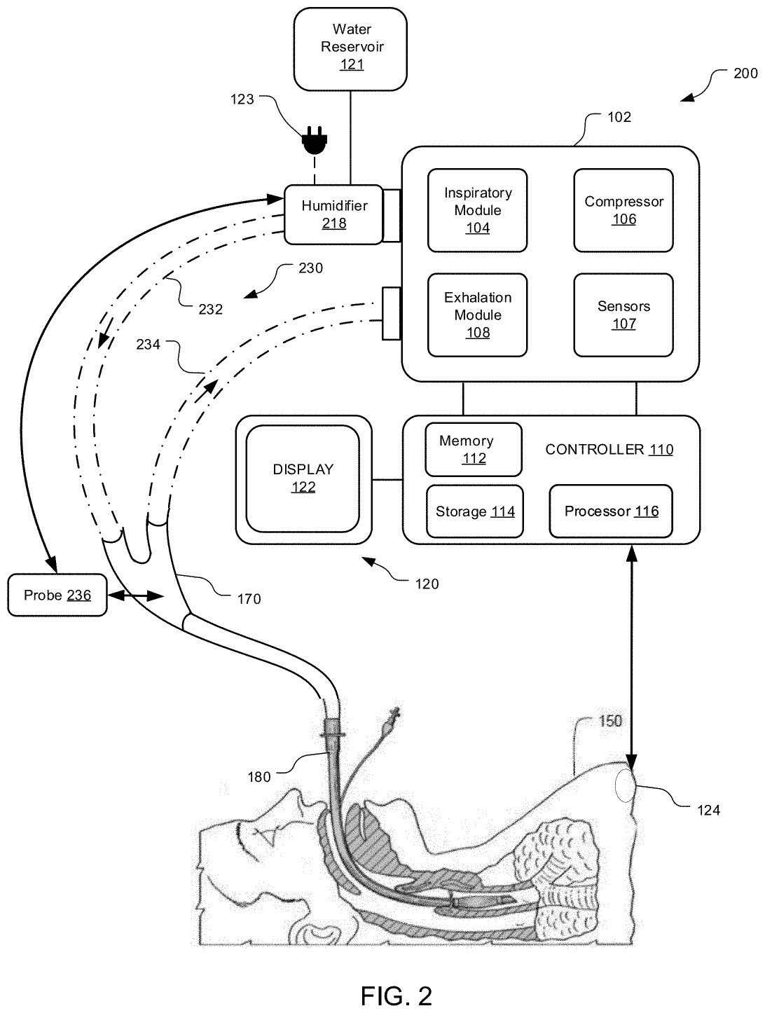

In some aspects, as illustrated by FIG. 1, humidifier 118 may be a stand-alone device, including a controller and processors for monitoring and regulating humidity of the breathing gases, as well as including an independent gas flow sensor. The humidifier 118 may also be in communication with the sensors 107 and/or may receive the values measured or determined by the sensors 107. The data from the sensors 107 may be provided to the humidifier 118 directly or may be provided from the ventilator 100. In example depicted in FIG. 2, humidifier 118 may be installed outside of the ventilator 100 near inspiratory port 125 and may be independently powered via power interface 123. In some aspects, humidifier 118 may be integrated with the ventilator 100 and may include a controller and processors for monitoring and regulating humidity of the breathing gases, but may not include an independent gas flow sensor. In still other aspects, humidifier 118 may be integrated with and controlled by ventilator 100 via controller 110, may not comprise an independent gas flow sensor, and may also be powered by ventilator 100 (not shown). Whether the humidifier 118 is integrated with the ventilator or is a stand-alone device, the humidifier 118 may access a water supply via water reservoir 121, which may be independent of (as shown) or integrated with ventilator 100. Additionally, the water supply accessed by humidifier 118 may be filtered by a water filter (not shown). In some cases, a medicine may be dissolved in the water supply, e.g., where the water supply is an intravenous (IV) bag.

In examples where a heating tube 119 is included in addition to the heated surfaces discussed herein that protrude into the conduit, the heating tube 119 may form a short conduit (e.g., two to five inches long) downstream of humidifier 118 (shown) and upstream of patient interface 180. Alternatively, heating tube 119 may be integrated into humidifier 118 and may form a short conduit within or coupled to the inspiratory limb 132. Heating tube 119 may comprise a thermally conductive material, such as aluminum, silver, copper, or other suitable metal or alloy (which, in some cases may be thinly plated with nickel to prevent corrosion), and a heating element. In some aspects, the heating element may be a heater blanket surrounding the thermally conductive material of heating tube 119. The heating element may generate thermal energy via any suitable means, e.g., electrical, chemical, or otherwise, and may deliver the thermal energy to the thermally-conductive material via any suitable means (e.g., via an external sleeve or blanket, internal or external wiring, etc.). In aspects, the heating element may heat quickly, e.g., in less than one minute, and may be controlled by humidifier 118 and/or ventilator 100 to achieve a desired temperature. As illustrated, heating tube 119 is in fluid communication with the inspiratory limb 132 of the ventilation tubing system 130. In this way, heating tube 119 contacts air or liquid in the flow path for maintaining a desired or target humidity of the breathing gases and preventing rainout in the ventilation tubing system 130. In some aspects, a second heating tube (not shown) may be placed on the exhalation side of the wye fitting 170 in order to maintain a desired humidity of exhaled gases and to prevent rainout in the exhalation limb 134 of the ventilation tubing system 130. In other aspects, the heating tube 119 may be omitted entirely.

The humidifier 118 may also include a controller (similar to controller 110) with a memory (similar to memory 112), one or more processors (similar to processors 116), storage (similar to storage 114), a display (similar to display 122) and/or other components of the type commonly found in command and control computing devices similar to the ones described above for the ventilator 100. In some cases, when humidifier 118 includes one or more of the above-described components of command and control computing devices, the humidifier 118 may be integrated with ventilator 100; in other cases, the humidifier 118 may be a stand-alone unit that is communicatively coupled to ventilator 100. As used herein, communicatively or operatively coupled refers to any wired or wireless communication infrastructure configured for receiving and/or transmitting commands, data, measurements, or other information. In some cases, whether the humidifier 118 is integrated with the ventilator 100 or is a stand-alone unit, the humidifier may be independently powered via power interface 123.

When humidifier 118 includes one or more of the above-described components of command and control computing devices (not shown), the humidifier memory includes non-transitory, computer-readable storage media that stores and/or encodes software (such as computer executable instruction) that is executed by the humidifier processor and which controls the operation of the humidifier 118. In an aspect, the humidifier memory includes one or more solid-state storage devices such as flash memory chips. In an alternative aspect, the humidifier memory may be mass storage connected to the humidifier processor through a mass storage controller (not shown) and a communications bus (not shown). Although the description of computer-readable media contained herein refers to a solid-state storage, it should be appreciated by those skilled in the art that computer-readable storage media can be any available media that can be accessed by the humidifier processor. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

FIG. 2 is a diagram illustrating a second aspect of an exemplary ventilator 200 connected to a human patient 150. Similar to ventilator 100, ventilator 200 includes a pneumatic system 102 for circulating breathing gases to and from patient 150 via a ventilation tubing system, which couples the patient 150 to the pneumatic system 102 via a patient interface 180 (e.g., endotracheal tube, as shown). Other than the components described below, the components of ventilator 200 are similarly described to the components of ventilator 100. Similar to ventilator 100, ventilator 200 is communicatively coupled to a humidifier 218. In the aspect illustrated by FIG. 2, humidifier 218 does not comprise heating tube 119 but is communicatively coupled to a heating circuit 230 and/or a probe 236.

Heating circuit 230 may comprise a heating inspiratory limb 232 and/or a heating exhalation limb 234. Heating circuit 230 may comprise a heating element (depicted by dashed lines) that is in contact with a substantial portion of the patient circuit, including a heating inspiratory limb 232 and/or a heating exhalation limb 234. The heating element may be independent and may surround (e.g., as a heater blanket) a traditional, disposable patient circuit to form heating circuit 230. In this case, the heating element may be non-disposable and capable of sterilization between patients; or the heating element may itself be disposable. Alternatively, the heating element may be integrated (e.g., wired) into a custom, disposable patient circuit to form heating circuit 230. The heating element may generate thermal energy via any suitable means, e.g., electrical, chemical, or otherwise, and may deliver the thermal energy to heat the patient circuit via any suitable means (e.g., via an external sleeve or blanket, internal or external wiring, etc.). In aspects, the heating element may heat quickly, e.g., in one minute or less, and may be controlled by humidifier 218, probe 236, and/or ventilator 100 to achieve a desired temperature.

As illustrated, heating circuit 230 includes heating inspiratory limb 232 (depicted by dashed lines) and heating exhalation limb 234 (depicted by dashed lines) and is in substantial fluid communication with breathing gases and exhalation gases to regulate temperature and humidity in the heating circuit 230. The purpose of heating the inspiratory limb is to heat the humidified breathing gases in order to control a temperature of the breathing gases at the wye fitting (e.g., between 32 and 42 degrees C.), to provide further evaporative heating power, and/or to prevent condensation of water on the inside walls of the inspiratory limb.

The purpose of heating the exhalation limb is to heat exhalation gases to prevent condensation from forming on the inside walls, so the temperature in the heating exhalation limb 234 may be maintained at a level just above the dew point of the exhaled gases (for example maintained at 44 degrees C.). In other examples, heating circuit 230 may comprise heating inspiratory limb 232 without heating exhalation limb 234. In such an example, heating inspiratory limb 232 may regulate temperature of the humidified breathing gases and may prevent rainout in the heating inspiratory limb 232 as well as minimizing rainout in the patient and in the non-heated exhalation limb 134 (not shown). The temperature of the inspiratory limb 232 and the exhalation limb 234 may also be based on a measured temperature of the patient 150. For example, a temperature of the patient may be obtained from the thermometer 124, and the target temperature of the breathing gases entering the patient 150 may be set or adjusted based on the measured patient temperature.

Probe 236 may be communicatively coupled to or integrated into wye fitting 170 (depicted by a two-way arrow). In one example, probe 236 comprises a temperature sensor and/or humidity sensor (not shown) for monitoring the temperature and/or humidity of the constituents (e.g., breathing gas and water) flowing through heating circuit 230. In another example, probe 236 is communicatively coupled to a temperature sensor and/or humidity sensor (not shown) associated with the wye fitting 170 for monitoring the temperature and/or humidity of the constituents (e.g., breathing gas and water) flowing through heating circuit 230. The temperature and/or humidity sensor is similar to temperature and/or humidity sensor 107, as described above. In further aspects, probe 236 is communicatively coupled to humidifier 118 (depicted by a two-way arrow) and may provide feedback to humidifier 218 regarding the temperature and/or humidity of breathing gases flowing to patient 150 and/or exhalation gases flowing back to the ventilator 200. For example, a temperature and humidity of breathing gases flowing to the patient 150 may be measured by the probe 236, and the temperature and humidity of the breathing gases exhaled from the patient 150 may be measured by the probe 236. Based on the feedback from probe 236, humidifier 218 may adjust an amount of water delivered to the flow path and/or may adjust an amount of heat delivered by the heating element to heating circuit 230. In some examples, the first probe and/or other sensors may be positioned on an inspiratory side of the wye to measure characteristics of the delivered breathing gases and a second probe or other sensors may be positioned on an expiratory side of the wye to measure the characteristics of the exhaled breathing gases. In other examples, the probe 236 is configured to measure the characteristics of the delivered breathing gases on the inspiratory side of the wye during breath delivery (e.g., an inhalation phase of a breath) and measure characteristics of the exhaled breathing gases during exhalation by the patient (e.g., an exhalation phase of the breath).

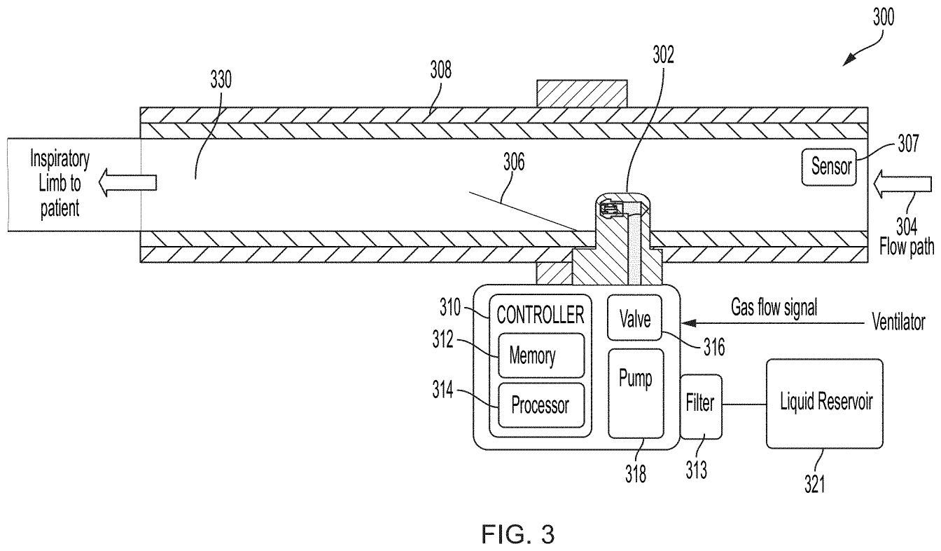

FIG. 3 is a partial cross-sectional schematic diagram illustrating an example humidifier 300, which may be similar to humidifier 118 or humidifier 218, discussed above. The humidifier 300 includes a liquid-injection nozzle 302 positioned in a conduit 308 and in a flow path 304 of a ventilator (similar to ventilator 100 or ventilator 200, discussed above above) during ventilation of a patient 150. The nozzle 302 may be configured to inject liquid (e.g., water and/or medicine) in one of a variety of patterns, including a jet, a full cone, a hollow cone, a fan shape, etc. The liquid may be injected as a stream of liquid or as atomized droplets. The properties of the injected liquid (e.g., stream versus droplet along with droplet size) may be based on the pressure of the liquid that is injected, the frequency at which the liquid is injected, and/or the size and configuration of the apertures in the nozzle 302. For instance, in an example the tip of the nozzle 302 may include one or more small holes or apertures that causes pressurized water to atomize when passing through the small holes. In other examples, the atomizer may include a hole or aperture for providing a jet of water. An elongated aperture may also or alternatively be included to provide the fan-shaped pattern of the injected water. In some examples, the nozzle 302 may include a micro-perforated membrane to inject liquid through the plurality of micro-perforations.

The humidifier 300 includes a heated surface 306, which may be a ramped surface as depicted in FIG. 3. The heated surface 306 may include a heating element and a thermally conductive material, such as aluminum, silver, copper, or other suitable metal or alloy (which, in some cases may be thinly plated with nickel to prevent corrosion). The heating element may generate thermal energy via any suitable means, e.g., electrical, chemical, or otherwise, and may deliver the thermal energy to the heated surface 306 via any suitable means (e.g., via an external sleeve or blanket, internal or external wiring, etc.).

As illustrated in FIG. 3, the heated surface 306 protrudes into and is positioned within the conduit 308 and exposed to the flow path 304 of the breathing gases. The heated surface 306 is positioned such that liquid injected from the nozzle 302 impinges the heated surface 306. The heated surface 306 vaporizes the liquid that impinges the heated surface 306. The evaporated liquid then mixes with the breathing gases in the flow path 304 to form humidified breathing gases 330. The humidified breathing gases 330 are carried into an inspiratory limb of a breathing circuit, and the humidified breathing gases 330 are ultimately inhaled by the patient. The heated surface 306 may heat quickly, e.g., in one minute or less, and may be controlled by humidifier 300 and/or ventilator 100 to rapidly achieve a desired temperature of the breathing gases within the conduit 308. As such, ventilator 100 and/or humidifier 300 require very little start up time for humidifying the breathing gas. In some examples, the conduit 308 may also be heated to also provide evaporation of liquid that contacts the inner walls of the conduit 308. For instance, liquid may bounce of splatter off of the heated surface 306 and contact the walls of the conduit 308.

The nozzle 302 is positioned to inject liquid directly into the flow path 304 of the breathing gases, and those breathing gases may exhibit variable initial humidity levels before entering the humidifier 300. For instance, where the breathing gas source is dry, such as from bottled gases, hospital wall gases, or gases from a compressor with dryer, then a greater amount of water may need to be injected into the breathing gas stream than would be the case, for example, if the breathing gas source is from a blower-based system that provides gases at an ambient humidity level. In examples where the humidifier 300 is integrated with the ventilator, the flow path 304 may originate within the pressure generating system and the gas inlet to the humidifier may be at the inspiratory port of the pressure generating system. Alternatively, where the humidifier 300 is a stand-alone device, the gas inlet to the humidifier 300 downstream from the pressure generating system but upstream from the wye 170 or the patient interface 180, as illustrated in FIG. 1.

In some aspects, the temperature of the heated surface 306 is maintained using closed-loop control by a controller 310 (or controller 110 of ventilator 100) to a level whereby the liquid ejected from the nozzle 302 is vaporized, and a temperature of the humidified breathing gases 330 is regulated to maintain the water vapor in the breathing gases delivered to the patient at a user-selected humidity. For instance, in examples without a heated inspiratory limb, for a target temperature of the delivered breathing gases of 37 degrees C., the humidified breathing gases leaving the humidifier may be about 45 degrees C. to account for cooling in the inspiratory limb of the patient circuit. In other aspects, the temperature of the heated surface 306 is significantly hotter than needed for vaporization in order to raise the temperature of the humidified breathing gases 330 to a desired temperature sufficient to maintain the water vapor in the breathing gases at a user-selected humidity when cooling occurs in the ventilation tubing system.

In some aspects, the humidifier 300 also includes a liquid reservoir 321, a liquid pump 318 and a valve 316, which are in fluid communication with the nozzle 302. For example, the liquid pump 318 pumps liquid from the liquid reservoir 321 towards the nozzle 302 through valve 316. The liquid pump 318 may be outside of the flow of ventilator gases. Accordingly, portions of the humidifier 300 that are exposed to the flow of gases may be separated from the pump 318 for cleaning. The pump 318 may also be capable of pumping fluid through the fluid line without components of the pump 318 coming into fluidic contact with the fluid. For instance, the pump 318 may be a tube pump, such as a peristaltic pump, a full-press ring pump, a mid-press ring pump, or other pump configured to pump a fluid without components of the pump coming into fluidic contact with the fluid. In such examples, the pump 318 may not contact the breathing gases or the fluid that is being pumped, which results in the pump remaining relatively clean and not necessarily requiring sterilization between patients.

The liquid reservoir 321, such as an intravenous (IV) bag of distilled water or other suitable liquid supply, supplies liquid at ambient pressure to the pump 318. In some cases, a medication may be dissolved in the liquid reservoir 321, e.g., dissolved in the intravenous (IV) bag.

An outlet of the pump 318 may be directed to the valve 316. In some aspects, the valve 316 is a fast-response solenoid valve that delivers the pressurized liquid from the pump 318 to the nozzle 302. In other examples, the valve 316 may be omitted and control of the fluid to the nozzle 302 may be controlled directly by the pump 318. For instance, activation of the pump causes liquid to flow to the nozzle 302, and deactivation of the pump 318 reduces the pressure of the liquid against the nozzle 302. In such examples, the nozzle 302 may include a membrane that allows fluid to be injected only at pressures above a pressure threshold. Thus, activating the pump 318 causes the fluid pressure to exceed the threshold and liquid to be injected from the nozzle 302. When the pump 318 is deactivated, the pressure of the liquid drops as the liquid is injected through the nozzle 302 until the liquid pressure is below the pressure threshold and liquid substantially ceases to flow through the membrane.

The controller 310 may include memory 312 and at least one processor 314. Controller 310 may be operative to receive an inspiratory flow command from the ventilator (e.g., ventilator 100) and may command valve 316 and/or pump 318 to deliver an amount of fluid, such as water or medicine, sufficient to maintain a user-selected relative humidity of the breathing gases. The amount of fluid may be calculated to be sufficient to maintain the user-selected relative humidity of the breathing gases and/or to deliver a prescribed amount of the medicine based on a concentration of the medicine in the fluid. In aspects, a concentration of the medicine in the fluid may be adjusted based on the amount of water calculated to maintain the desired humidity. In other aspects, as detailed above, humidifier 300 may not include a controller and valve 316 and/or pump 318 may be controlled by the ventilator (e.g., ventilator 100).

As an example, controller 310 may command valve 316 and/or pump 318 using pulse width modulation (PWM) or some other suitable driving method to provide “bursts” of water to the nozzle 302. In these aspects, the duration and timing of bursts (as controlled by the opening and closing of the valve 316 and/or activating and deactivating the pump 318) provides a prescribed amount of pressurized liquid to the nozzle 302. These controlled bursts or pulses allow the nozzle 302 to inject a specific amount of liquid to the heated surface 306, thereby preventing or reducing over or under humidification as well as delivering a prescribed amount of a dissolved medicine, if desired.

In some examples, the width of the electric pulses that trigger the bursts of water may be less than 200 milliseconds, 100 milliseconds, less than 50 milliseconds, and/or between 5-50 milliseconds. For instance, the burst of liquid may last 5-50 milliseconds. In some examples, such as where a hollow cone atomizer is used for the nozzle 302, the pressures of the liquid may be quite high and in excess of 250 pounds per square inch (PSI), 300 PSI, and/or 350 PSI. In other examples, where different water injection patterns are used (e.g., fan shape, full cone, etc.), lower pressures may be used, such as less than 200 PSI, between 50-100 PSI, and/or between 50-150 PSI. For instance, a flat or fan shaped spray pattern may allow for lower pressures to be used as compared to a hollow-cone shaped spray pattern. The lower pressure requirements allow for a larger variety of pumps to be implemented, such as a peristaltic pump as discussed above.

Each burst of water delivers a precise amount of water into the patient circuit. Thus, based on the configuration of the nozzle 302 (e.g., aperture size, number, and configuration), the burst duration, and the liquid pressure, the amount of liquid delivered to the patient circuit may be calculated and/or determined. Accordingly, the amount of liquid from the humidifier that is delivered to the patient may be determined on a continuous basis, such as on a breath-by-breath basis. The amount of water may also be determined in real-time and based on ventilation. For instance, a first amount of water may be injected during an inhalation phase of a breath and a second amount of water may be injected during an exhalation phase of the breath.

Additionally, the nozzle 302 may be configured to spray or inject liquid in spray patterns of small water droplets at a low flow rate. The low flow rate further enables the nozzle 302 to prevent or reduce over humidification by having a higher resolution of the amount of liquid that is injected into the system.

In some aspects, to achieve a desired humidity, the water flow rate is dependent on flow rate of breathing gases flowing through the humidifier 300. For instance, an average water flow rate as low as 0.04 ml/min may be delivered at a gas flow rate of 1 liters/min; whereas an average water flow rate as high as 9 ml/min may be delivered at a gas flow of 200 liters/min. Accordingly, the atomizer may be designed to have the capability of providing a fluid flow rate of at least 9 ml/min so it can accommodate a gas flow rate of 200 liters/min. Thus, to accommodate lower gas flow rates, the solenoid valve may be pulsed with shorter durations and/or longer intervals between pulses to deliver less liquid flow. In this case, the nozzle 302 may deliver pulses of liquid at 30 ml/min timed and spaced to provide an average liquid flow rate of 1 ml/min.

In general, the nozzle 302 may be configured to deliver a liquid flow rate from 0.1 to 40.0 ml/min to breathing gases in the flow path 304 exhibiting a gas flow rate from 1 to 200 liters/min. These fluid flow rates are provided as examples and not meant to be limiting. Other suitable liquid flow rates for use with the humidifier 300 will be appreciated by a person of skill in the art in light of this disclosure. In some aspects, the humidifier 300 also includes a water filter 313. The water filter 313 prevents small debris from entering the pump 318, the valve 316, and/or the nozzle 302 by filtering out any debris from the liquid reservoir 321. As illustrated, the water filter 313 is located upstream of the pump 318, the valve 316, and the nozzle 302. In other aspects, the water filter 313 may be located downstream of the pump 318 and upstream of the valve 316 and the nozzle 302.

As illustrated, the humidifier 300 may also include a temperature sensor and/or humidity sensor 307 located in flow path 304 upstream of the nozzle 302. In other aspects, a temperature senor and/or a humidity sensor 307 may be located within the ventilator (e.g., associated with the inspiratory module 104) upstream of the nozzle 302 but separate and distinct from the humidifier 300. In these aspects, the temperature sensor and/or a humidity sensor 307 is not part of the humidifier 300 but is part of the ventilator (e.g., ventilator 100). The temperature sensor and/or humidity sensor 307 may be communicatively coupled to humidifier 300 and may provide temperature and/or humidity measurements to controller 310, which may then command the heated surface 306 (and/or a heating element of a heated breathing circuit or inspiratory limb, not shown) to maintain a desired temperature and/or humidity of the breathing gases flowing through flow path 304. Alternatively, the temperature sensor and/or humidity sensor 307 may provide temperature and/or humidity measurements to controller 110 of ventilator 100 and ventilator 100 may then command the heated surface 306 (and/or heating element of a heated breathing circuit or inspiratory limb, not shown) to maintain a desired temperature and/or humidity of the breathing gases flowing through flow path 304. In the example depicted, humidifier 300 does not comprise a gas flow sensor and is integrated with the ventilator (e.g., ventilator 100 or ventilator 200). In other examples, however, a gas flow sensor may be incorporated into the humidifier 300.

In some aspects, a second nozzle (not shown) may be provided in the flow path 304 passing through the humidifier 300. In some examples, both the first nozzle and second nozzle may configured to inject water. In other examples, the second nozzle may be designed to deliver a different liquid than the first nozzle, such as a medicine. The second nozzle may then be configured based on the fluid characteristics of the different liquid (e.g., medicine or medicines) to be delivered. For instance, when medicines are not water-soluble, these medicines may be significantly more viscous than water, and therefore the dimensions of the nozzle may need to be adjusted to appropriately atomize the medicine. Depending on the fluid characteristics, this second nozzle may may have a different spray shape than the first nozzle. For instance, the second atomizer may generate a full cone droplet pattern rather than a fan pattern.

Where medicine is dispersed by the second nozzle, a full cone or fan beam spray pattern may be preferable so that more of the medicine is provided into the breathing gases rather than on the sidewalls of the breathing circuit. Further, there may be no need to heat or evaporate the medicine, and therefore the second nozzle may be configured such that the fluid injected from the second nozzle does not impinge on the heated surface 306. For example, the second nozzle may be positioned downstream of the heated surface 306. In other examples, the second nozzle may extend from a different position of the interior sidewall of the conduit 308. For instance, in the example depicted, the first nozzle 302 protrudes from the bottom of the conduit 308, and the second nozzle may protrude from the top of the conduit 308. In other examples, a second heated surface may be provided for the second nozzle such that fluid injected by the second nozzle impinges on the second heated surface. The second heated surface may have similar characteristics as the other heated surfaces discussed herein.

The second nozzle may use the same type of reservoir, pumping and valve system, as described below. Alternatively, depending on the fluid characteristics of the medicine, the second nozzle may require adjustments to the reservoir, pumping, and/or valve system as appropriate for the fluids and the pressures used. In aspects, a medicine dissolved in a biologically compatible solvent is delivered to the second nozzle via a suitable valve and/or pumping system. Similar to the first atomizer, the second nozzle disperses the medicine-solvent solution in small droplets into the flow path. Depending on the location of the second nozzle with respect to the heated surface 306, and the fluid characteristics of the medicine-solvent solution, the small droplets may or may not be vaporized by the humidifier 300. However, it is contemplated that small droplets of the medicine-solvent may deliver a prescribed amount of the medicine to the breathing gases without requiring vaporization. In some examples, the second atomizer may be a removable plug-in device, e.g., connected via an access port in the humidifier housing that may be covered when not in use.

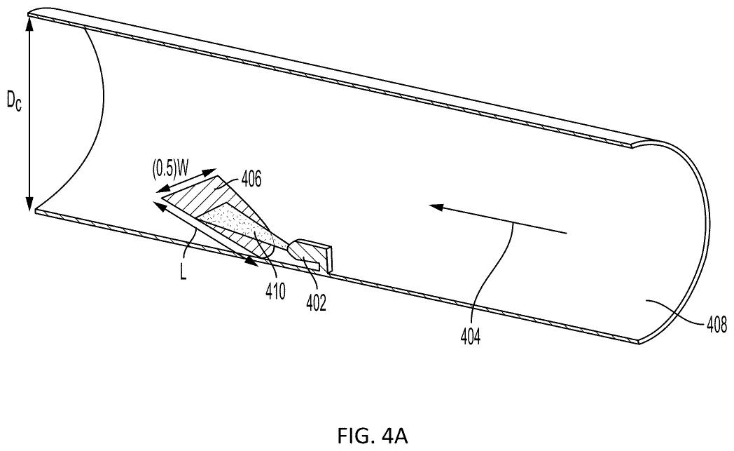

FIG. 4A depicts a perspective view of a partial, cross-sectional diagram illustrating a configuration for a nozzle 402 and a heated surface 406 of a humidifier. In the example depicted, the nozzle 402 is positioned within a conduit 408 that receives as flow of breathing gases from a ventilator in the direction of the flow path 404. The nozzle 402 protrudes from an inner surface of the conduit 408 towards the center of the conduit. The nozzle 402 is configured to inject a liquid 410 into the conduit and towards the heated surface 406. In the example depicted, the nozzle 402 is configured to inject the fluid in substantially the same direction as the flow path 404. In other examples, the nozzle 402 may be configured to inject the liquid 410 in a direction opposite to the flow path 404.

The nozzle 402 is also configured to inject the liquid 410 in a fan beam shape, which is substantially two-dimensional. For instance, the fan beam expands in substantially a single plane (as compared to a hollow or full cone pattern that expands in three-dimensions). The injected liquid 410 impinges the heated surface 406 where the fluid is evaporated or vaporized.

In the examples depicted, the heated surface 406 is a ramped surface the extends or protrudes into the conduit 408 at an angle. By using a ramped configuration, the evaporated water is directed into the center of the flow path 404 of breathing gases. For instance, the injected liquid 410 travels at a velocity towards the heated surface 406 where it is evaporated and deflected by the ramp towards the center of the conduit 408. As will be appreciated, the flow velocity of the breathing gases may be greatest near the center of the conduit 408. Thus, by the directing the evaporated fluid towards the center of the conduit 408, better mixing of the evaporated fluid and the breathing gases may occur, which may also further reduce the likelihood of rainout downstream of the humidifier (e.g., between the humidifier and the patient 150.)

The heated surface 406 may be characterized by its length (L) and width (W). In the cross section depicted, the width is depicted as half the width (W) as only half of the conduit is depicted in FIG. 4A. In the example depicted, the length (L) is the maximum length of the heated surface 406 and the width (W) is the maximum width of the heated surface 406. In other examples, the width (W) and/or the length (L) may be the average width and average length of the heated surface 406. The length (L) may be between 10-50 mm, and the width (W) of at least 30% of the inner diameter (D_{C}) of the conduit 408. For instance, the width (W) may be between 50%-100% or 60%-80% of the inner diameter (D_{C}) of the conduit 408.

FIG. 4B depicts a side view of the configuration depicted in FIG. 4A. Additional details and dimensions of the configuration can be seen in in FIG. 4A. For instance, the heated surface 406 extends into the conduit 408 at a ramp angle (α). The ramp angle (α) may be between 20-90 degrees, between 30-60 degrees, or between 10-50 degrees. Accordingly, the heated surface 406 crosses or intersects the flow path of breathing gases. For instance, the heated surface 406 at least partially faces the flow of breathing gases, and the portion of the heated surface 406 on which the injected liquid 410 impinges is non-parallel to the flow of breathing gases.

The ramped heated surface 406 also has a height (H_{R}), which may be the maximum height of the heated surface 406 from the interior surface of the conduit from which the heated surface 406 protrudes. The height (H_{R}) may be between the 10%-70% of the inner conduit diameter (D_{C}). The ramp angle (α) may also be based on the height (H_{R}) of the heated surface 406 and the length (L) of the heated surface 406.

The nozzle 402 also protrudes from the interior surface of the conduit 408. The nozzle 402 has a height (H_{N}), which is the height of the center point from where the fluid is injected by the nozzle 402. The height (H_{N}) of the nozzle 402 may be based on the height of the heated surface 406 and the position of the heated surface. For instance, the height of the nozzle 402 may be configured such that substantially all the fluid (e.g., at least 95%) injected from the nozzle 402 impinges the heated surface 406. Thus, the height (H_{N}) of the nozzle 402 may be related to, or based on, the height (H_{R}) of the heated surface 406 (or vice versa). For instance, the height (H_{N}) of the nozzle 402 may be less than the height of the heated surface 406. As an example, the height (H_{N}) of the nozzle 402 may be between 20%-80% or 40-60% of the height (H_{R}) of the heated surface 406. In addition, by utilizing a ramped heated surface 406 rather than (or in addition to) a heating tube, the height of the nozzle 402 need not be centered in the conduit 408. Rather, the nozzle 402 may be placed much closer to the interior surface of the conduit 408. Further, the use of the heated surfaces discussed herein, rather than a heating tube, allows for the injected liquid 410 to be vaporized without having to travel across the entire diameter of the conduit before reaching the inner walls heating tube.

The heated surface 406 is positioned a distance (D_{NS}) from the nozzle 402. The distance (D_{NS}) may be measured from the tip of the nozzle 402 from where the liquid 410 leaves the nozzle 402 to a target point on the heated surface 406 where a center line of the liquid 410 where the fluid impinges the heated surface 406. The center line of the liquid 410 is the center of the spray pattern (e.g., cone, fan, jet, etc.). In other examples, the distance (D_{NS}) may be measured from the tip of the nozzle 402 from where the liquid 410 leaves the nozzle 402 to a geometric center of the heated surface 406. In some examples, the distance (D_{NS}) may be between 10%-40% of the inner conduit diameter (D_{C}).

The distance (D_{NS}) may be based on the spray shape and characteristics of the injected liquid 410. For instance, for an expanding spray shape (e.g., cone or fan), the distance (D_{NS}) may be set such that substantially all the fluid (e.g., at least 95%) injected from the nozzle 402 impinges the heated surface 406. The distance (DNs) may also be set based on stream and droplet size characteristics for injected fluid. For instance, the formation and dispersion of droplets is dependent on the viscosity of the fluid, the size of the apertures, the pressure of the liquid, and/or the frequency of the injections and may generally be controlled by the Rayleigh instability principles. Thus, the distance (D_{NS}) may be set such that the injected liquid 410 has formed as droplets prior to impinging the heated surface 406.

FIG. 4C depicts another example configuration with a different nozzle angle (β). The nozzle angle (β) determines the direction in which the liquid 410 is injected into the conduit 408. The nozzle angle (β) is the angle between an axial line 412 and a center line of the liquid 410 injected from the nozzle 402. The axial line 412 is a line that is parallel with a longitudinal axis of the conduit 408, which may also be parallel to the flow path 404. The nozzle angle (β) may be between about 0-30 degrees or 10-30 degrees. The nozzle angle (β) may be upward (e.g., nozzle 402 pointing up towards the conduit wall opposite the wall from which the nozzle 402 protrudes) or downward (e.g., nozzle 402 pointing down towards the conduit wall from which the nozzle 402 protrudes). Where the nozzle angle (β) is upward, as depicted in FIG. 4C, the nozzle height (H_{N}) may be lower, such as between 10%-40% of the heated surface height (H_{R}).

FIG. 4D depicts another example configuration with a curved heated surface 406. The configuration depicted in FIG. 4D is substantially similar to the configuration in FIGS. 4A-4B with the exception that the heated surface 406 is curved rather than planar. The curved surface may provide some additional surface area and ability to catch injected liquid 410 injected from the nozzle 402. The curved surface may also provide additional deflection of the injected water to encourage mixing the vapor with the breathing gases.

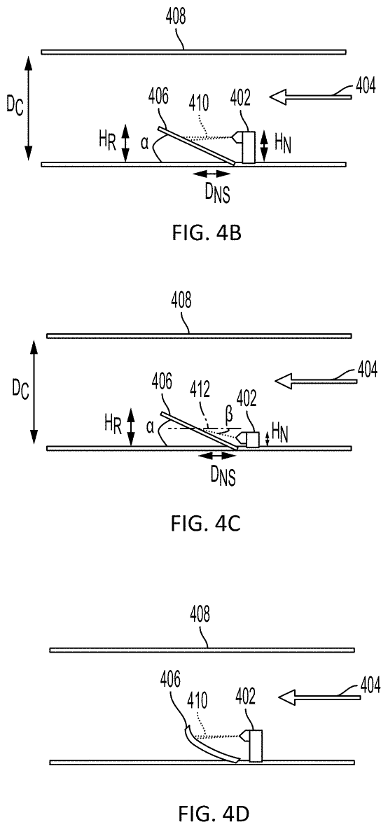

FIG. 5A depicts an example configuration for a nozzle 502 and a heated surface 506 of a humidifier. Similar to the above configurations, the nozzle 502 injects fluid 510 such that the fluid 510 impinges the heated surface 506 where the fluid is evaporated. In the example depicted in FIG. 5A, the nozzle 502 injects the fluid 510 in a direction that is in the same direction as the flow path 504. The nozzle 502 injects the fluid 510 in a full cone or fan beam spray pattern. The heated surface 506 is a planar surface that is located near the center of the conduit 508. The heated surface 506 have a variety of shapes, such as rectangular, square, circular, pentagonal, hexagonal, etc. Smaller securing mechanisms or standoffs (not depicted) may be incorporated to attach or secure the heated surface 506 in the position towards the center of the conduit 508. The length (L) of the heated surface 506 may be between 20-85% or 40-60% of the diameter of the conduit 508.

FIG. 5B depicts another example configuration for a nozzle 502 and a heated surface 506 of a humidifier. The configuration in FIG. 5B is substantially the same as the configuration in FIG. 5A with the exception that the flow path 504 is in the opposite direction. As such, in FIG. 5B, with respect to the flow path 504 of breathing gases, the nozzle 502 is positioned downstream from the heated surface 506.

FIG. 5C depicts another example configuration for a nozzle 502 and a heated surface 506 of a humidifier. The configuration in FIG. 5C is substantially the same as the configuration in FIG. 5A with the exception that the heated surface 506 is angled rather than perpendicular to the flow path 504. The heated surface 506 may be angled between 10-70 degrees from the vertical or perpendicular position shown in FIG. 5A.

FIG. 5D depicts another example configuration for a nozzle 502 and a heated surface 506 of a humidifier. The configuration in FIG. 5C is substantially the same as the configuration in FIG. 5A with the exception that the injected fluid 510 is injected as a jet spray pattern rather than a fan beam or full cone spray pattern.

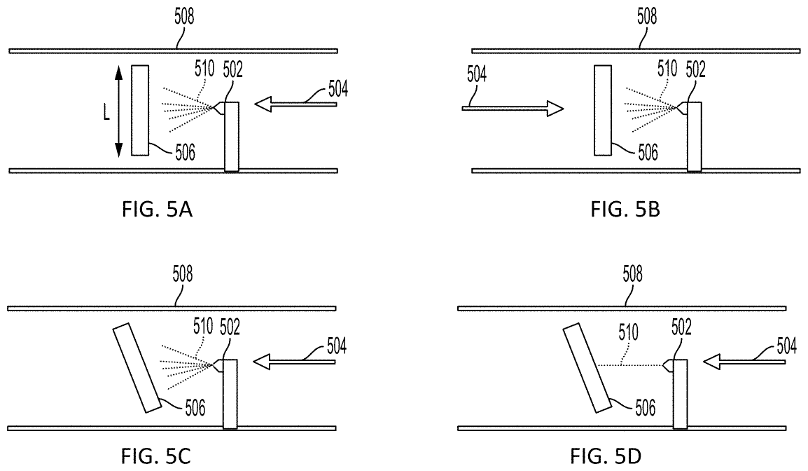

FIG. 6A depicts another example configuration for a nozzle 602 and a heated surface 606 of a humidifier. The heated surface 606 in FIG. 6A has a substantially parabolic shape with a concave, or interior, portion of the shaped facing the nozzle 602 such that the liquid 610 injected from the nozzle 602 impinges the interior surface of the parabolic heated surface 606. The heated surface 606 may be mounted to the conduit through mounting structures (not shown). The parabolic shape of the heated surface 606 helps capture substantially all of the liquid 610 that is injected from the nozzle. In the example depicted, the nozzle 602 injects liquid 610 towards in the same direction as the flow path 604, but in other examples, the nozzle 602 may inject liquid 610 in a direction opposite the flow path 604. In yet other examples, the heated surface 606 may be oriented in the opposite direction such that the exterior, or convex, portion of the parabolic heated surface 606 faces the nozzle 602.

In some examples, the heated surface 608 may be perforated to include small through holes (e.g., perforations) through the heated surface 608. The perforations may be sized such that the breathing gas is able to pass through the perforations but the liquid droplets may not. For instance, it may be desirable to prevent the liquid droplets from traveling downstream and reaching the patient's lungs in their liquid form. However, it may also be beneficial to reduce the effect the heating surface has on the flow of breathing gases. Thus, incorporating small holes or perforations may provide for better airflow of breathing gases while still preventing liquid droplets from passing through the holes. In some examples, the heated surface 606 may be made from a mesh with small openings that substantially prevent the liquid droplets from passing through the mesh. In other examples, portions of the heated surface 606 may be made from a material that allows breathing gases and/or water vapor to pass through the material but not allow for liquid to pass through. Such a material may be a waterproof breathable material. The waterproof breathable material may be incorporated into one or more of the holes or perforations of the heated surface 606.

FIG. 6B depicts another example configuration for a nozzle 602 and a heated surface 606 of a humidifier. In the example configuration in FIG. 6B, the heated surface 606 is substantially spherical with an opening facing the nozzle 602 such that the liquid 610 injected from the nozzle 602 may be received in the interior portion of the spherical heated surface 606. In other examples, the spherical heated surface 606 may substantially encapsulate the nozzle 602. The spherical heated surface 606 may also include perforations or holes to allow breathing gases to flow through the heated surface 606 and mix with the evaporated liquid in a same or similar manner as discussed above.

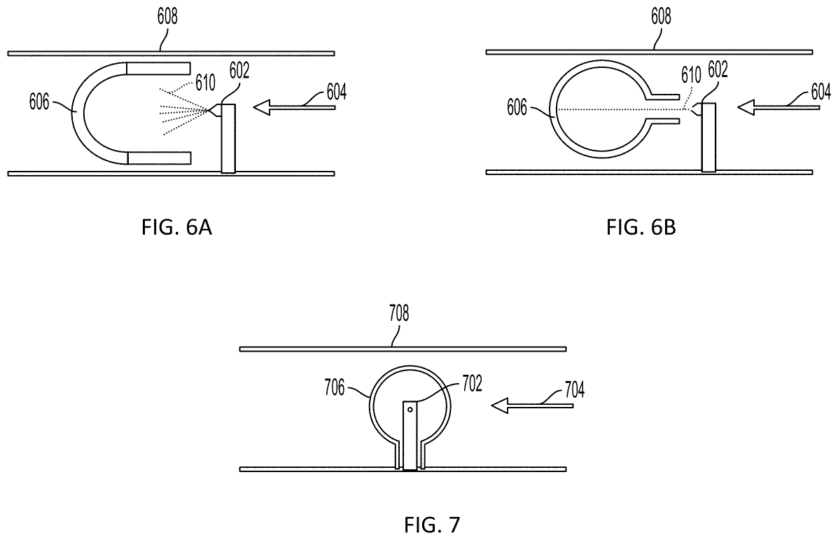

FIG. 7 depicts another example configuration for a nozzle 702 and a heated surface 706 positioned within a conduit 708 of a humidifier. Unlike the other examples above, the nozzle 702 is configured to inject the fluid in a direction that is substantially orthogonal to the direction of the flow path 704 of the breathing gases. The fluid, however, is still injected such that it impinges a heated surface 706, which is a spherical heated surface 706 in the example depicted. In such a configuration, a portion of the heated surface 706 is positioned upstream (with reference to the flow of breathing gases) from the nozzle 702 and another portion of the heated surface is positioned downstream of the nozzle 702. The spherical heated surface 706 may be perforated or include a series of holes similar to the examples discussed above.

While some different shapes and configurations of heated surfaces are described above, it should be appreciated that a variety of shapes of the heated surface may be utilized, such as parabola-shaped, bowl-shaped, conic, frustroconic, substantially cylindrical, partially cylindrical, etc. In examples each of the above shapes, the heated surfaces have a portion of the heated surface that is not parallel to the flow of breathing gases, which is in contrast to the walls of the conduit which are parallel to the flow of breathing gases. For instance, the heated surfaces have a portion of the surface that is at least partially orthogonal (e.g., not parallel) to the flow of breathing gases. As an example, more than 50% or 80% of the heated surface facing the nozzle may be angled (e.g., at least partially orthogonal) to the flow of breathing gases or a central axis of the conduit.

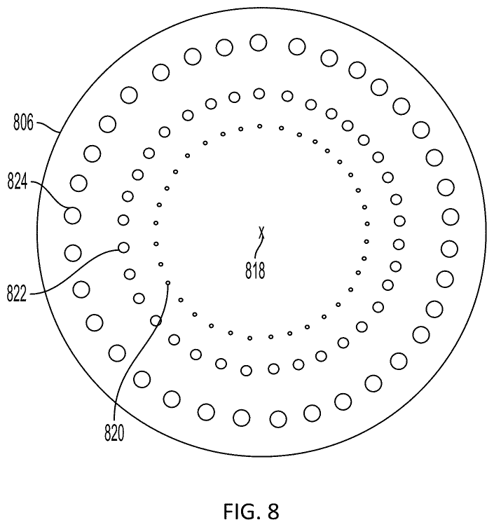

FIG. 8 depicts an example perforation pattern for a heated surface 806. The heated surface 806 includes a target location 818 where the center line of the injected fluid impinges the heated surface 806. The heated surface 806 includes a plurality of through holes or perforations 820, 822, 824. The diameter of the through holes increase as their respective radial position away from the target location 818 increases. For instance, the inner ring of perforations 820 have the smallest diameter, the middle ring of perforations 822 have a larger diameter, and the outer ring of perforations 824 have the largest diameter. The central portion of the heated surface 806, however, may not include any perforations. The central portion may be the central 10-30% of the surface area of the heated surface 806. Such a perforation pattern of increasing perforation size while leaving a central portion of the heating surface un-perforated allows for vaporization of injected fluid, reduction of fluid droplets that pass through the heated surface, and reduction in negative impacts on the flow of breathing gases.

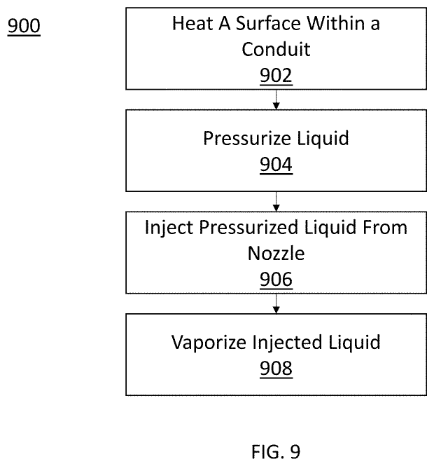

FIG. 9 depicts an example method 900 for humidifying breathing gases from a ventilator. At operation 902, a surface protruding into a conduit for carrying breathing gases is heated to a temperature. The temperature may be based on a desired temperature of the breathing gases and a temperature sufficient to vaporize an injected fluid, as discussed above. The heated surface may be any of the heated surfaces discussed herein.

At operation 904, liquid is pressurized by a pump, and at operation 906, the pressurized liquid is injected from the nozzle into the conduit. The pressurized liquid is injected such that it impinges the heated surface. As discussed above, injection of the pressurized liquid may be controlled by activating and deactivating a pump and/or a valve of the humidifier. Such activation and deactivation may be controlled though pulse width modulation techniques or other techniques that allow for short bursts of pressurized liquid to be injected. At operation 908, the injected fluid is vaporized by the heated surface and the evaporated form of the injected liquid mixes with the breathing gases to form humidified breathing gases.

Those skilled in the art will recognize that the methods and systems of the present disclosure may be implemented in many manners and as such are not to be limited by the foregoing exemplary aspects and examples. In other words, functional elements being performed by a single component or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known. Thus, myriad software/hardware/firmware combinations are possible in achieving the functions, features, interfaces, and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter.

This disclosure describes some embodiments of the present technology with reference to the accompanying drawings, in which only some of the possible embodiments were shown. Other aspects may, however, be embodied in many different forms and should not be construed as limited to the embodiments set forth herein. Rather, these embodiments were provided so that this disclosure was thorough and complete and fully conveyed the scope of the possible embodiments to those skilled in the art. Further, as used herein and in the claims, the phrase “at least one of element A, element B, or element C” is intended to convey any of: element A, element B, element C, elements A and B, elements A and C, elements B and C, and elements A, B, and C. Further, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the appended claims.

What is claimed is:

1. A humidification system, comprising:
a conduit for carrying breathing gases;
a pump to pressurize a liquid for injection into breathing gases;
a liquid-injection nozzle protruding at least partially into the conduit and configured to inject liquid, pressurized by the pump, into the conduit; and
a heated surface protruding into the conduit and positioned to vaporize the liquid injected by the nozzle, wherein the heated surface crosses a flow path of the breathing gases flowing through the conduit.

2. The humidification system of claim 1, wherein the heated surface is a ramped surface protruding from an inner wall of the conduit.

3. The humidification system of claim 2, wherein the ramped surface has a ramp angle from the conduit wall between 10 degrees and 50 degrees.

4. The humidification system of claim 2, wherein the ramped surface has a width of at least 30% of a diameter of the conduit.

5. The humidification system of claim 1, wherein the nozzle is configured to inject the liquid in one of a jet spray pattern or fan beam spray pattern.

6. The humidification system of claim 1, wherein the heated surface is substantially orthogonal to the flow of breathing gases.

7. The humidification system of claim 1, wherein the heated surface is perforated to allow the breathing gases to flow through the perforations.

8. The humidification system of claim 1, wherein the heated surface has a concave portion facing the flow of breathing gases and the nozzle, wherein the concave portion is positioned to receive and vaporize the injected liquid.

9. The humidification system of claim 1, wherein the heated surface has a convex portion facing the flow of gases and the nozzle, wherein the convex portion is positioned relative to the nozzle to receive and vaporize the injected liquid.

10. The humidification system of claim 1, wherein the heated surface is at least one of: parabola-shaped, bowl-shaped, conic, frustroconic, substantially cylindrical, partially cylindrical.

11. The humidification system of claim 1, wherein a portion of the heated surface is positioned downstream of the nozzle and another portion of the heated surface is positioned upstream of the nozzle.

12. The humidification system of claim 1, wherein the heated surface substantially encapsulates the nozzle.

13. A humidification system, comprising:
a conduit for carrying breathing gases;
a pump to pressurize a liquid for injection into breathing gases;
a liquid-injection nozzle protruding at least partially into the conduit and configured to inject liquid, pressurized by the pump, into the conduit; and
a ramped heated surface protruding into the conduit from an interior surface of the conduit and positioned to vaporize at least a portion of the liquid injected by the nozzle, wherein the ramped heated surface protrudes into the conduit at a ramp angle between 20-90 degrees.

14. The humidification system of claim 13, wherein the ramped heated surface has a height between 10%-70% of an inner diameter of the conduit, wherein the height of the ramped heated surface is the maximum height of the heated surface from the interior surface of the conduit.

15. The humidification system of claim 14, wherein the nozzle has a nozzle height between 20%-80% of the height of the ramped heated surface, wherein the nozzle height is a height above the interior surface of the conduit to a center point of the nozzle from where the liquid is injected by the nozzle.

16. The humidification system of claim 13, wherein the ramped surface has a width of at least 30% of a diameter of the conduit.

17. The humidification system of claim 13, wherein the nozzle is configured to inject the liquid in a direction opposite a direction of a flow path of the breathing gases.

18. The humidification system of claim 17, wherein the ramped heated surface is positioned upstream, with respect to the flow path of breathing gases, from the nozzle.

19. The humidification system of claim 13, wherein the nozzle has an upward nozzle angle between 10-30 degrees.

20. A method for humidifying ventilator-delivered breathing gases, the method comprising:
heating a heated surface protruding into a conduit, wherein the heated surface is non-parallel with the flow of breathing gases through the conduit;
pressurizing, by a pump, a liquid;
injecting the pressurized liquid through a nozzle protruding into the conduit, wherein the injected liquid impinges the heated surface; and
vaporizing, by the heated surface, the injected liquid.
